(19) United States
(12) Patent Application Publication
(10) Pub. No.: US 2025/0170094 A1
(43) Pub. Date: May 29, 2025

(54) ANTI-INFLUENZA VIRUS PHARMACEUTICAL FORMULATIONS

(71) Applicant: Hefei Cosource Pharmaceuticals Co., Ltd, Hefei (CN)

(72) Inventors: Jun Zhang, Hefei (CN); Dengjun Chen, Hefei (CN); Yuting Jin, Hefei (CN); Liuyu Shi, Hefei (CN); Xiao Wang, Hefei (CN); Xiaoling Wang, Hefei (CN); Lixia Ke, Hefei (CN); Weichen Zhou, Hefei (CN); Xiaorong Lu, Hefei (CN)

(73) Assignee: Hefei Cosource Pharmaceuticals Co., Ltd, Hefei (CN)

(21) Appl. No.: 18/812,369

(22) Filed: Aug. 22, 2024

(30) Foreign Application Priority Data
Nov. 27, 2023 (CN) 202311598906.3

(51) Int. Cl. A61K 31/351; A61K 31/196; A61K 47/02; A61K 47/12; A61K 47/18

(52) U.S. Cl. CPC A61K 31/351; A61K 31/196; A61K 47/02; A61K 47/12; A61K 47/18

(57) ABSTRACT

The present disclosure provides a pharmaceutical formulation, wherein raw materials for formulating the pharmaceutical formulation comprise: an active ingredient which is one selected from the group consisting of peramivir, zanamivir, laninamivir, and hydrates or solvates thereof; a carboxylic acid anion donor which is at least one selected from the group consisting of pamoic acid and hydroxynaphthoic acid; and a first pH regulator which is an alkaline pH regulator.

### CROSS-REFERENCE TO RELATED APPLICATIONS

This application claims priority to and benefits of Chinese Patent Application No. 202311598906.3 filed before the China National Intellectual Property Administration on Nov. 27, 2023, the entire contents of which are incorporated herein by reference

### TECHNICAL FIELD

The present application relates to the field of pharmaceuticals, and more specifically, to an anti-influenza virus pharmaceutical formulation, a preparation method thereof, and use of the anti-influenza virus pharmaceutical formulation in the manufacture of a medicament for preventing or treating a disease caused by an influenza virus.

### BACKGROUND

Globally, influenza virus infections cause massive morbidity and mortality every year Influenza viruses have become an important source of sudden novel viral infectious diseases due to their high degree of mutability and susceptibility to human bodies, and the risk of a global pandemic of novel influenza viruses is increasing, which poses a great challenge to the public health system. Moreover, reports of drug resistance of existing anti-influenza virus drugs are increasing in recent years, and under this background, there is an urgent need for new therapeutic approaches and regimens for influenza virus infections with high efficacy and high drug loading.

Peramivir (chemical name: (1S,2S,3R,4R)-3-[(1S)-1-(acetylamino)-2-ethylbutyl]-4-guanidino-2-hydroxycyclopentanecarboxylic acid) is a neuraminidase inhibitor which, like oseltamivir and zanamivir, can bind to the active site of neuraminidase of an influenza virus to prevent the spread of the virus. Peramivir exhibits unique in vitro anti-drug resistance compared with oseltamivir and zanamivir, and certain viral variants resistant to oseltamivir and zanamivir are sensitive to peramivir

Peramivir has been marketed in a number of countries, including the United States, Japan, China, etc., for the prevention and treatment of influenza A or B virus infections. The FDA-approved specification for peramivir injection for marketing in the United States is 200 mg/20 ml (10 mg/ml), the specifications approved for marketing in Japan are 300 mg/60 ml (5 mg/ml) and 150 mg/15 ml (10 mg/ml), and the specifications approved for marketing in China are 300 mg/100 ml (3 mg/ml) and 150 mg/100 ml (1.5 mg/ml).

Because of the high polarity and low oral bioavailability of peramivir, only formulations for intravenous administration of peramivir are available on the market, and the administration route of peramivir is relatively single and can hardly meet the increasingly severe clinical needs. Peramivir has an extremely low solubility, and the highest concentration of a peramivir injection currently used in clinic is merely 10 mg/ml; for a clinical dose of 600 mg, a volume of at least 60 ml of the drug solution is required, which limits the development, mode of administration, and clinical use of peramivir formulations.

### SUMMARY OF INVENTION

In a first aspect, the present application provides a pharmaceutical formulation, wherein raw materials for formulating the pharmaceutical formulation comprise: an active ingredient which is one selected from the group consisting of peramivir, zanamivir, laninamivir and hydrates or solvates thereof; a carboxylic acid anion donor which is at least one selected from the group consisting of pamoic acid and hydroxynaphthoic acid; and a first pH regulator which is an alkaline pH regulator.

In some embodiments, the hydroxynaphthoic acid is one or more selected from the group consisting of 2-hydroxy-1-naphthoic acid, 3-hydroxy-1-naphthoic acid, 4-hydroxy-1-naphthoic acid, 5-hydroxy-1-naphthoic acid, 6-hydroxy-1-naphthoic acid, 1-hydroxy-2-naphthoic acid, 2-hydroxy-3-naphthoic acid, and 8-hydroxy-2-naphthoic acid; and optionally, the hydroxynaphthoic acid is 2-hydroxy-3-naphthoic acid.

In some embodiments, the first pH regulator is one or more selected from the group consisting of sodium hydroxide, potassium hydroxide, diethylamine, triethylamine. ethylenediamine, sodium bicarbonate, sodium carbonate, trometamol, and meglumine.

In some embodiments, a molar ratio of the active ingredient to a carboxyl group in the carboxylic acid anion donor is 1:(0.5 to 3), or 1:(0.7 to 2).

In some embodiments, the raw materials further comprise a cation donor; optionally, the cation donor is one or more selected from the group consisting of inorganic salts of magnesium ions, calcium ions, zinc ions, potassium ions, and sodium ions, trometamol, meglumine, and ethylenediamine; or the cation donor is one or more selected from the group consisting of magnesium chloride, magnesium sulfate, calcium chloride, and zinc chloride, and the cation donor is the same as or different from the first pH regulator.

In some embodiments, a molar ratio of the active ingredient: a carboxyl group in the carboxylic acid anion donor: a cation in the cation donor is 1:(0.5 to 3):(0 to 5), or 1:(0.7 to 2):(0.2 to 2).

In some embodiments, the raw materials comprise: the active ingredient which is one selected from the group consisting of peramivir, zanamivir, laninamivir and hydrates or solvates thereof; the carboxylic acid anion donor which is one or more selected from the group consisting of 2-hydroxy-3-naphthoic acid, 1-hydroxy-2-naphthoic acid, and pamoic acid; the first pH regulator which is one or more selected from the group consisting of sodium hydroxide, potassium hydroxide, diethylamine, ethylenediamine, triethylamine, sodium bicarbonate, sodium carbonate, trometamol, and meglumine; and optionally, a cation donor which is one or more selected from the group consisting of magnesium chloride, magnesium sulfate, calcium chloride, and zinc chloride.

In some embodiments, the raw materials further comprise a second pH regulator which is one or more selected from the group consisting of sodium hydroxide, potassium hydroxide, diethylamine, triethylamine, ethylenediamine, sodium bicarbonate, sodium carbonate, trometamol, meglumine, hydrochloric acid, sodium citrate, citric acid, sodium dihydrogen phosphate, disodium hydrogen phosphate, and phosphoric acid.

In some embodiments, the pharmaceutical formulation is an aqueous pharmaceutical formulation or a lyophilized pharmaceutical formulation, optionally, the aqueous pharmaceutical formulation is a liquid inhalation formulation, an aqueous injection solution, a spray, or an aerosol; optionally, the aqueous pharmaceutical formulation is an oral aerosol inhalation formulation.

In some embodiments, the pharmaceutical formulation is an aqueous pharmaceutical formulation, wherein an concentration of the active ingredient is up to 100 mg/ml, and optionally the concentration of the active ingredient ranges from 20 mg/ml to 100 mg/ml; or the pharmaceutical formulation is a lyophilized pharmaceutical formulation, and in an aqueous solution reformulated from the lyophilized pharmaceutical formulation with water, a concentration of the active ingredient is up to 100 mg/ml, and optionally the concentration of the active ingredient ranges from 20 mg/ml to 100 mg/ml.

In some embodiments, the aqueous pharmaceutical formulation or the aqueous solution reformulated from the lyophilized pharmaceutical formulation with water has a pH value of 5.5 to 8.5, or 6.5 to 8.0.

In some embodiments, the pharmaceutical formulation is a lyophilized pharmaceutical formulation containing a lyophilization excipient; optionally, the lyophilization excipient is one or more selected from the group consisting of sodium chloride, glucose, glycine, cysteine, and lysine; or the lyophilization excipient is one selected from the group consisting of sodium chloride, glucose, and glycine; optionally, the lyophilized pharmaceutical formulation contains 0 wt % to 5 wt % of the lyophilization excipient.

In some embodiments, a route of administration of the pharmaceutical formulation is selected from the group consisting of intravenous administration, oral aerosol inhalation, nasal administration, subcutaneous administration, intradermal administration, and intramuscular administration, or is oral aerosol inhalation.

In a second aspect, the present application provides a preparation method of the pharmaceutical formulation of the first aspect, comprising the following steps: (1) dissolving the carboxylic acid anion donor and the alkaline pH regulator in water; and (2) adding the active ingredient into the aqueous solution obtained in step (1).

In some embodiments, the preparation method further comprises: (3) adding the cation donor after the active ingredient is added.

In some embodiments, the preparation method further comprises: by using a second pH regulator, adjusting a pH value of the aqueous solution obtained in step (1) to 7 or higher, or to 7 to 9, and/or adjusting a pH value of the aqueous solution obtained in step (2) to 5.5 to 8.5, or to 6.5 to 8.0, wherein the second pH regulator is one or more selected from the group consisting of sodium hydroxide, potassium hydroxide, diethylamine, triethylamine, ethylenediamine, sodium bicarbonate, sodium carbonate, trometamol, meglumine, hydrochloric acid, sodium citrate, citric acid, sodium dihydrogen phosphate, disodium hydrogen phosphate, and phosphoric acid.

In some embodiments, the preparation method further comprises a step of aseptically filling and/or lyophilizing the aqueous solution obtained in step (2) to obtain a lyophilized pharmaceutical formulation.

In a third aspect, the present application provides use of the pharmaceutical formulation of the first aspect in the manufacture of a medicament for preventing or treating a disease caused by an influenza virus, e.g., an acute respiratory infectious disease caused by influenza A virus or influenza B virus.

### BRIEF DESCRIPTION OF DRAWINGS

The embodiments illustrated herein are further described below with reference to the accompanying drawings, which, however, are intended merely to enable those skilled in the art to better understand the present application and are not intended to limit the scope of the present application.

FIG. 1 shows drug plasma concentration-time profiles in the plasma of SD rats for a peramivir inhalation solution and a peramivir injection solution prepared according to Formulation 5 of Example 1.

FIG. 2 shows drug concentration-time profiles in the lung tissue of SD rats for a peramivir inhalation solution and a peramivir injection solution prepared according to Formulation 5 of Example 1.

FIG. 3 shows drug concentration-time profiles in the plasma and the lung tissue of SD rats for a peramivir inhalation solution prepared according to Formulation 5 of Example 1.

FIG. 4 shows drug concentration-time profiles in the plasma and the lung tissue of SD rats for a peramivir injection solution prepared according to Formulation 5 of Example 1.

### DESCRIPTION OF EMBODIMENTS

Hereinafter, the invention concepts of the present application will be further elucidated according to specific embodiments. However, the listed specific embodiments are for illustrative purposes only and are not intended to limit the scope of the present application. Those skilled in the art will recognize that a specific feature in any of the embodiments below may be used in any other embodiment as long as it does not depart from the invention concepts described herein.

Unless otherwise indicated, all numbers indicating sizes, quantities, and physicochemical properties of features used in the specification and claims should be understood to be modified by the term “about” in all instances. Thus, unless stated to the contrary, the numerical parameters set forth in the specification and the appended claims are approximations, which may be varied by those skilled in the art under the teachings disclosed herein in order to seek for the desired characteristics. The utilization of a numerical range represented by endpoints includes all numbers within the range and any range within the range, for example, 1 to 5 includes 1, 1.1, 1.3, 1.5, 2, 2.75, 3, 3.80, 4, and 5, etc.

Peramivir, zanamivir, and laninamivir are zwitterions in which carboxyl groups and guanidino groups between the molecules form salt bridges, thereby facilitating aggregation into crystal cells that are not easily soluble.

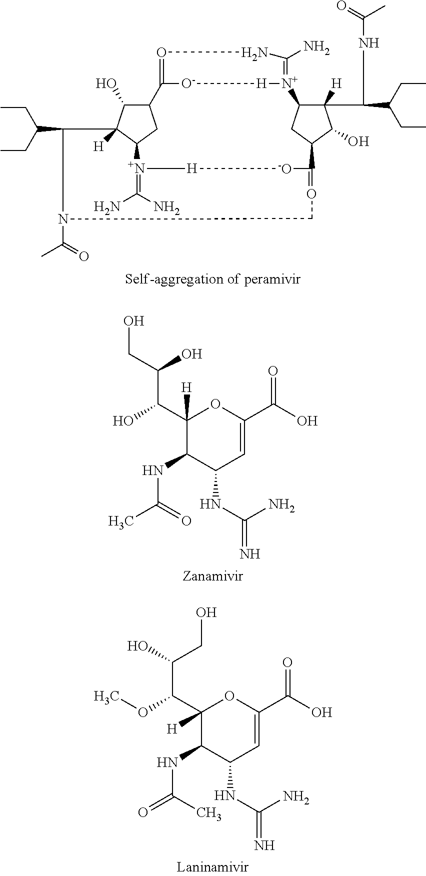

Peramivir is used clinically at an injection dose of 600 mg, and 3% to 9% of the AUC migrates to the upper respiratory tract fluid according to the human tissue migration data. If an inhalation formulation is developed, it is possible that only 10% of the injection dose, i.e., an inhalation dose of 60 mg, is required. The aerosol delivery rate is generally about 25%, and the volume of the inhalation formulation is generally about 2 ml. It is known that peramivir has a low solubility (the solubility is about 59 mg/ml at pH 1.0, and ≤20 mg/ml at pH≥5), and the existing solubility of peramivir cannot meet the requirements for preparing the inhalation formulation. The volume of administration limits the development, mode of administration, and clinical use of peramivir formulations, and peramivir cannot be solubilized using conventional 15 solubilization techniques such as addition of organic solvents, surfactants, etc.

Without being bound by theory, a pharmaceutical formulation in the present application comprises a carboxylic acid anion donor (e.g., pamoic acid or hydroxynaphthoic acid), a carboxyl group of which preferentially interacts with a guanidino group on the peramivir molecule, thereby preventing intermolecular polymerization of peramivir, and greatly increasing the solubility of peramivir. The pharmaceutical formulation of the present application may further comprise a cation donor that interacts with a carboxyl group on the peramivir molecule to further enhance the solubility of peramivir and to improve the stability of the composition.

In one aspect, the present application provides a pharmaceutical formulation, wherein raw materials for formulating the pharmaceutical formulation comprise: an active ingredient which is one selected from the group consisting of peramivir, zanamivir, laninamivir, and hydrates or solvates thereof; a carboxylic acid anion donor which is one or more selected from the group consisting of pamoic acid and hydroxynaphthoic acid; and a first pH regulator which is an alkaline pH regulator.

In some embodiments, the hydroxynaphthoic acid is one or more selected from the group consisting of 2-hydroxy-1-naphthoic acid, 3-hydroxy-1-naphthoic acid, 4-hydroxy-1-naphthoic acid, 5-hydroxy-1-naphthoic acid, 6-hydroxy-1-naphthoic acid, 1-hydroxy-2-naphthoic acid, 2-hydroxy-3-naphthoic acid, and 8-hydroxy-2-naphthoic acid. In some embodiments, 2-hydroxy-3-naphthoic acid is used to provide the carboxylic acid anion. In some embodiments, pamoic acid is used to provide the carboxylic acid anion.

In some embodiments, the first pH regulator is one or more selected from the group consisting of sodium hydroxide, potassium hydroxide, diethylamine, triethylamine, ethylenediamine, sodium bicarbonate, sodium carbonate, trometamol, and meglumine. For example, the first pH regulator is one or more selected from the group consisting of sodium hydroxide, trometamol, and meglumine.

In some embodiments, the pamoic acid and the hydroxynaphthoic acid are insoluble in water, and the first pH regulator is added to enable the formation of pamoate salts and hydroxynaphthoate salts, and to adjust the pH to reach visual clarification with a measured pH 7 to 9.

In some embodiments, the pharmaceutical formulation of the present application is formulated in water from the raw materials of the present application: the active ingredient, the carboxylic acid anion donor, and the first pH regulator. In some embodiments, a molar ratio of the active ingredient to a carboxyl group in the carboxylic acid anion donor is 1:(0.5 to 3), optionally 1:(0.7 to 2). The ratio of hydroxynaphthoic acid anions may affect the solubility and stability of the product. When the content of the hydroxynaphthoic acid anions is too low, the resulting pharmaceutical formulation cannot be clarified or maintains clarification for a short time after resolubilization. When the content of the bydroxynaphthoic acid anions added is too high, a large amount of alkali is added to dissolve the hydroxynaphthoic acid, and the final product is adjusted back to a physiological pH value with an acid, which results in a relatively high osmotic pressure of the final product that needs to be ensured to be within a physiological range.

In some embodiments, the raw materials of the pharmaceutical formulation of the present application further comprise a cation donor. In some embodiments, the cation donor is one or more selected from the group consisting of inorganic salts of magnesium ions, calcium ions, zinc ions, potassium ions, and sodium ions, trometamol, meglumine, and ethylenediamine. In some embodiments, the cation donor is one or more selected from the group consisting of magnesium chloride, magnesium sulfate, calcium chloride, trometamol, and meglumine. In some embodiments, the cation donor is trometamol. Since trometamol can form hydrogen bonds with peramivir, and may have some chelation effects, trometamol has a better effect on increasing sample stability.

In some embodiments, the first pH regulator, e.g., trometamol and meglumine, also has an effect as a cation ligand, and thus the raw material of the cation donor used may be the same as or different from the raw material of the first pH regulator.

The submicroscopic structure of the embodiments of the present application can be determined by dynamic light scattering, and it can be observed that the aqueous solution sample or the sample obtained by lyophilization and resolubilization according to the embodiments of the present application has a nanopolymer structure. The particle size can be controlled in the range of several nanometers (0.5 nm to 10 nm) when the selected cations are added and in the range of tens to hundreds of nanometers (20 nm to 200 nm) when the cations are not added. The larger the particle size is after lyophilization and resolubilization, the greater the likelihood of aggregation is, and the more unstable the sample is. The addition of the cations may further improve the solubility stability of the active ingredient in the formulated aqueous solution or in the aqueous solution obtained by lyophilization and resolubilization.

In some embodiments, the raw materials of the pharmaceutical formulation of the present application comprise: the active ingredient which is one selected from the group consisting of peramivir, zanamivir, laninamivir and hydrates or solvates thereof; the carboxylic acid anion donor which is one or more selected from the group consisting of pamoic acid and hydroxynaphthoic acid; the first pH regulator; and the cation donor, wherein a molar ratio of the active ingredient: a carboxyl group in the carboxylic acid anion donor: a cation in the cation donor is 1:(0.5 to 3):(0 to 5). In some embodiments, the molar ratio of the active ingredient: a carboxyl group in the carboxylic acid anion donor: a cation in the cation donor is 1:(0.7 to 2):(0.2 to 2).

In some embodiments, the raw materials of the pharmaceutical formulation of the present application comprise: the active ingredient which is one selected from the group consisting of peramivir, zanamivir, laninamivir, and hydrates or solvates thereof; the carboxylic acid anion donor which is one or more selected from the group consisting of 2-hydroxy-3-naphthoic acid, 1-hydroxy-2-naphthoic acid, and pamoic acid; the first pH regulator which is one or more selected from the group consisting of sodium hydroxide, potassium hydroxide, diethylamine, ethylenediamine, triethylamine, sodium bicarbonate, sodium carbonate, trometamol, and meglumine; and optionally, a cation donor which is one or more selected from the group consisting of magnesium chloride, magnesium sulfate, calcium chloride, and zinc chloride.

In some embodiments, the raw materials of the pharmaceutical formulation of the present application comprise: the active ingredient which is one selected from the group consisting of peramivir, zanamivir, laninamivir, and hydrates or solvates thereof; the carboxylic acid anion donor which is one or more selected from the group consisting of 2-hydroxy-3-naphthoic acid, 1-hydroxy-2-naphthoic acid, and pamoic acid; the first pH regulator which is one or more selected from the group consisting of trometamol, meglumine, and sodium hydroxide; and optionally, a cation donor which is one or more selected from the group consisting of magnesium chloride, magnesium sulfate, calcium chloride, and zinc chloride.

In some embodiments, the raw materials of the pharmaceutical formulation of the present application comprise: peramivir trihydrate; 2-hydroxy-3-naphthoic acid, 1-hydroxy-2-naphthoic acid, or pamoic acid; trometamol; and magnesium sulfate.

As an embodiment, the raw materials of the pharmaceutical formulation of the present application comprise: peramivir trihydrate; 2-hydroxy-3-naphthoic acid; trometamol; and magnesium sulfate; optionally, an amount ratio of peramivir trihydrate: 2-hydroxy-3-naphthoic acid: trometamol: magnesium sulfate is 69.9 mg:34.4 mg to 86 mg:38.1 mg:4.8 mg; and optionally, the pharmaceutical formulation is an aqueous solution with a concentration of peramivir of 60 mg/ml.

As an embodiment, the raw materials of the pharmaceutical formulation of the present application comprise: peramivir trihydrate; 1-hydroxy-2-naphthoic acid; trometamol; and magnesium sulfate; optionally, an amount ratio of peramivir trihydrate:1-hydroxy-2-naphthoic acid:trometamol:magnesium sulfate is 69.9 mg:68.8 mg:38.1 mg:4.8 mg; and optionally, the pharmaceutical formulation is an aqueous solution with a concentration of peramivir of 60 mg/ml.

As an embodiment, the raw materials of the pharmaceutical formulation of the present application comprise: peramivir trihydrate; pamoic acid; trometamol; and magnesium sulfate; optionally, an amount ratio of peramivir trihydrate:pamoic acid:trometamol:magnesium sulfate is 69.9 mg:56.8 mg to 142 mg:38.1 mg:4.8 mg; and optionally, the pharmaceutical formulation is an aqueous solution with a concentration of peramivir of 60 mg/ml.

In some embodiments, the raw materials of the pharmaceutical formulation of the present application comprise: peramivir trihydrate; pamoic acid; and meglumine; optionally, an amount ratio of peramivir trihydrate:pamoic acid:meglumine is 69.9 mg:63.9 mg:17.9 mg; and optionally, the pharmaceutical formulation is an aqueous solution with a concentration of peramivir of 60 mg/ml.

In some embodiments, the raw materials of the pharmaceutical formulation of the present application comprise: peramivir trihydrate; pamoic acid; meglumine; and magnesium sulfate, magnesium chloride, zinc chloride or anhydrous calcium chloride.

In some embodiments, the raw materials of the pharmaceutical formulation of the present application comprise: peramivir trihydrate; pamoic acid; meglumine; and magnesium sulfate; optionally, an amount ratio of peramivir trihydrate:pamoic acid:meglumine:magnesium sulfate is 69.9 mg:63.9 mg:17.9 mg:4.8 mg to 9.6 mg; and optionally, the pharmaceutical formulation is an aqueous solution with a concentration of peramivir of 60 mg/ml.

In some embodiments, the raw materials of the pharmaceutical formulation of the present application comprise: peramivir trihydrate; pamoic acid; meglumine; and magnesium chloride; optionally, an amount ratio of peramivir trihydrate:pamoic acid:meglumine:magnesium chloride is 69.9 mg:63.9 mg:17.9 mg:5 mg to 10 mg; and optionally, the pharmaceutical formulation is an aqueous solution with a concentration of peramivir of 60 mg/ml.

In some embodiments, the raw materials of the pharmaceutical formulation of the present application comprise: peramivir trihydrate; pamoic acid; meglumine; and zinc chloride; optionally, an amount ratio of peramivir trihydrate:pamoic acid:meglumine:zinc chloride is 69.9 mg:63.9 mg:17.9 mg:5.8 mg; and optionally, the pharmaceutical formulation is an aqueous solution with a concentration of peramivir of 60 mg/ml.

In some embodiments, the raw materials of the pharmaceutical formulation of the present application comprise: peramivir trihydrate; pamoic acid; meglumine; and anhydrous calcium chloride; optionally, an amount ratio of peramivir trihydrate:pamoic acid:meglumine:anhydrous calcium chloride is 69.9 mg:63.9 mg:17.9 mg:1.1 mg; and optionally, the pharmaceutical formulation is an aqueous solution with a concentration of peramivir of 60 mg/ml.

In some embodiments, the raw materials of the pharmaceutical formulation of the present application comprise: peramivir trihydrate, zanamivir, or laninamivir; pamoic acid; sodium hydroxide: and magnesium sulfate.

In some embodiments, the raw materials of the pharmaceutical formulation of the present application comprise: peramivir trihydrate; pamoic acid; sodium hydroxide: and magnesium sulfate; optionally, an amount ratio of peramivir trihydrate:pamoic acid:sodium hydroxide:magnesium sulfate is 46.6 mg to 116.5 mg:63.9 mg to 95.9 mg:14.6 mg: 4 mg; and optionally, the pharmaceutical formulation is an aqueous solution with a concentration of peramivir of 40 mg/ml to 100 mg/ml.

In some embodiments, the raw materials of the pharmaceutical formulation of the present application comprise: zanamivir; pamoic acid; sodium hydroxide: and magnesium sulfate; optionally, an amount ratio of zanamivir:pamoic acid:sodium hydroxide:magnesium sulfate is 24.3 mg:63.9 mg:14.6 mg:4 mg; and optionally, the pharmaceutical formulation is an aqueous solution with a concentration of zanamivir of 24.3 mg/ml.

In some embodiments, the raw materials of the pharmaceutical formulation of the present application comprise: laninamivir; pamoic acid; sodium hydroxide: and magnesium sulfate; optionally, an amount ratio of laninamivir:pamoic acid:sodium hydroxide:magnesium sulfate is 25.3 mg:63.9 mg:14.6 mg:4 mg; and optionally, the pharmaceutical formulation is an aqueous solution with a concentration of laninamivir of 25.3 mg/ml.

In some embodiments, the raw materials of the pharmaceutical formulation of the present application comprise: peramivir trihydrate; pamoic acid; sodium hydroxide; and magnesium chloride; optionally, an amount ratio of peramivir trihydrate:pamoic acid:sodium hydroxide:magnesium chloride is 58.2 mg:29.5 mg:14.6 mg:5 mg; and optionally, the pharmaceutical formulation is an aqueous solution with a concentration of peramivir of 50 mg/ml.

In some embodiments, the raw materials of the pharmaceutical formulation of the present application further comprise a second pH regulator which is one or more selected from the group consisting of the first pH regulator (sodium hydroxide, potassium hydroxide, diethylamine, triethylamine, ethylenediamine, sodium bicarbonate, sodium carbonate, trometamol, and meglumine), hydrochloric acid, sodium citrate, citric acid, sodium dihydrogen phosphate, disodium hydrogen phosphate, and phosphoric acid. The second pH regulator may be used to adjust the pH during the formulation of the pharmaceutical formulation.

In some embodiments, the pharmaceutical formulation of the present application is a (sterile) aqueous pharmaceutical formulation or (sterile) lyophilized pharmaceutical formulation. In some embodiments, the aqueous pharmaceutical formulation of the present application is a liquid inhalation formulation, an aqueous injection solution, a spray, or an aerosol. The pharmaceutical formulation of the present application can be prepared into various dosage forms by using conventional methods in the art, for example, a conventional mixing method, a dissolving method, a grinding method, a freeze drying method, etc.

In some embodiments, the aqueous pharmaceutical formulation of the present application is an oral aerosol inhalation formulation. Compared with intravenous and oral formulations, inhalation formulations have a high concentration in lesions and a low systemic exposure, which can reduce the dose of administration, enhance the efficacy, and reduce systemic toxic and side effects.

In some embodiments, the pharmaceutical formulation of the present application is an aqueous pharmaceutical formulation in which the concentration of the active ingredient is up to 100 mg/ml, and the high drug loading can inhibit the occurrence of viral resistance. In some other embodiments, the concentration of the active ingredient in the aqueous pharmaceutical formulation of the present application ranges from 20 mg/ml to 100 mg/ml. The concentration or loading of the active ingredient mentioned in this disclosure is calculated based on peramivir (C₁₅H₂₈N₄O₄), zanamivir (C₁₂H₂₀N₄O₇), or laninamivir (C₁₃H₂₂N₄O₇).

In some embodiments, the pharmaceutical formulation of the present application is a lyophilized pharmaceutical formulation, and in an aqueous soultion reformulated from the lyophilized pharmaceutical formulation with water, a concentration of the active ingredient is up to 100 mg/ml. In some other embodiments, in an aqueous soultion reformulated from the lyophilized pharmaceutical formulation with water, a concentration of the active ingredient ranges from 20 mg/ml to 100 mg/ml.

In some embodiments, the aqueous pharmaceutical formulation or the aqueous soultion reformulated from the lyophilized pharmaceutical formulation with water according to the present application has a pH value of 5.5 to 8.5, further 6.5 to 8.0.

In some embodiments, the lyophilized pharmaceutical formulation of the present application contains a lyophilization excipient. In some embodiments, the lyophilization excipient is one or more selected from the group consisting of sodium chloride, glucose, glycine, cysteine, and lysine. In some embodiments, the pharmaceutical formulation of the present application contains 0 wt % to 5 wt % of the lyophilization excipient.

In some embodiments, a solvent used for the formulation or clinical compatibility of the pharmaceutical formulation of the present application is selected from water for injection, physiological saline, glucose solutions, or physiologically acceptable buffers.

In some embodiments, a route of administration of the pharmaceutical formulation of the present application includes intravenous administration, oral aerosol inhalation, nasal administration, subcutaneous administration, intradermal administration, and intramuscular administration. For example, the route of administration of the pharmaceutical formulation of the present application is oral aerosol inhalation.

In some embodiments, the pharmaceutical formulation of the present application is an oral aerosol inhalation formulation, wherein the active ingredient is dosed at a dose of 20 mg to 800 mg, and an administration volume is not greater than 8 ml; or the active ingredient is dosed at a dose of 40 mg to 320 mg, and an administration volume is not greater than 4 ml; or the active ingredient is dosed at a dose of 60 mg to 240 mg, and an administration volume is not greater than 4 ml.

The pharmaceutical formulation of the present application can improve the antiviral efficacy of the active ingredient. For example, the oral aerosol inhalation formulation can be prepared into an aqueous formulation with a high drug loading, and a single dose that can be inhaled is up to 20 to 800 mg (based on an inhalation volume of 1 to 8 ml), thereby effectively inhibiting virus and avoiding drug failure caused by virus resistance, while improving patient compliance.

In some embodiments, the volume of the liquid inhalation formulation prepared by the present application can be reduced to 1 ml (100 mg/ml) due to the high drug loading, and the reduced volume can increase the sterility assurance level during the production process while reduce the scale and production costs; facilitate the production, transportation and storage processes, and further ensure the quality thereof; and reduce the infusion volume and time in clinical use and improve patient compliance.

In some embodiments, the pharmaceutical formulation of the present application has a long-acting effect in vivo that lasts at least 48 to 72 hours of absorption and distribution. The oil-water partition coefficient LogP[n-octanol/water] of peramivir is −1.16, indicating that peramivir has a strong hydrophilicity, but has a very small partition amount in n-octanol, i.e., a weak lipophilicity; peramivir itself is a compound having a strong polarity and poor membrane permeability, and can be retained in the lungs for a long time, so that the compound properties of peramivir itself make it have a certain long-acting effect. In addition, peramivir forms salts with cations, which increases the ionization of peramivir, and such complexes are positively charged at a physiological pH value, which can reduce the apparent permeability in the lung epithelial model, and increase the retention time of peramivir in the lungs, thereby achieving the long-acting effect. Moreover, the submicroscopic structure of the aqueous formulation provided in the present application has a nanopolymer structure, a larger steric hindrance of which itself has an effect of prolonging the time of action of peramivir. The aqueous formulation provided by the present application can realize an antiviral efficacy when administrated through oral aerosol inhalation route once within a week, which reduces the administration frequency, and greatly improves the compliance of the patient.

The present application provides an aqueous formulation having a high drug loading (up to 800 mg), and the drug can be completely dissolved by an injection water system in clinical use, thereby avoiding the use of auxiliary materials such as surfactants, solubilizers, and latent solvents to improve solubility, and at the same time avoiding toxic reactions such as hemolysis and irritation caused by surfactants and organic solvents.

In another aspect, the present application provides a preparation method of the above pharmaceutical formulation, comprising the following steps: (1) dissolving the carboxylic acid anion donor and the alkaline pH regulator in water; and (2) adding the active ingredient to the aqueous solution obtained in step (1).

In some embodiments, the preparation method of the present application further comprises: (3) adding the cation donor after the active ingredient is added.

In some embodiments, the preparation method of the present application further comprises, by using a second pH regulator, adjusting a pH value of the aqueous solution obtained in step (1) to 7 or higher, for example, 7 to 9, and/or adjusting a pH value of the aqueous solution obtained in step (2) to 5.5 to 8.5, for example, 6.5 to 8.0.

In some embodiments, the preparation method of the pharmaceutical formulation of the present application further comprises a step of aseptically filling and/or lyophilizing the aqueous solution obtained in step (2) to obtain a lyophilized pharmaceutical formulation.

In some embodiments, the present application provides a preparation method of the above pharmaceutical formulation, comprising:
step 1: weighing the carboxylic acid anion donor and the first pH regulator, adding water, stirring and shaking until dissolved, adding the active ingredient, stirring at 50° C. to 95° C. until dissolved to obtain a clear solution, and holding the temperature;
step 2 (optional): keeping stirring and cooling down to 40° C. or lower, adding the cation donor (preferably a metal cation donor), stirring and dissolving, and finalizing the volume; and
step 3: optionally adding a lyophilization excipient, and performing aseptic filling and/or lyophilization.

In some embodiments, the preparation method further comprises sterilization and filtration prior to the aseptic filling and/or lyophilization.

In some embodiments, the water used in step 1 is water for injection.

In some embodiments, the solution obtained in step 1 has a pH value of not less than 7, e.g., 7 to 9, and the solution obtained in step 2 has a pH value of 5.5 to 8.5, e.g., 6.5 to 8.0.

In some embodiments, in step 1, in addition to the use of the first pH regulator to dissolve the carboxylic acid anion donor in water, a second pH regulator may be used to adjust the pH to a range of 7 to 9; and/or a second pH regulator may be used in step 2 to adjust the pH to a suitable pH of 5.5 to 8.5, e.g., 6.5 to 8.0. The second pH regulator may be the same as or different from the first pH regulator.

In another aspect, the present application provides the use of the pharmaceutical formulation of the present application in the manufacture of a medicament for preventing or treating a disease caused by an influenza virus.

In another aspect, the present application provides a method of preventing or treating a disease caused by an influenza virus, comprising administering the pharmaceutical formulation of the present application to a subject (preferably, a mammal, e.g., a human being) in need thereof.

According to the results of in vivo pharmacokinetic experiments in rats, it is demonstrated that the pharmaceutical formulation of peramivir provided in the present application can enable enrichment of peramivir in the lungs after administration, has the effect of preventing or treating the disease caused by the influenza virus, and can be administrated by aerosol inhalation to realize the enrichment of the active drug in the lungs.

In some embodiments, the disease caused by the influenza virus to be prevented or treated by the pharmaceutical formulation of the present application is an acute respiratory infectious disease caused by an influenza A or B virus. The influenza virus includes the H1N1 and H3N2 subtypes of the influenza A viruses and the Victoria and Yamagata lineages of the influenza B viruses.

### EXAMPLES

Unless otherwise specified, the drugs or reagents used in the following examples are conventional and commercially available products. The raw material of peramivir used was peramivir trihydrate, manufactured by Hunan Jiudian Hongyang Pharmaceutical Co., Ltd.

### Comparative Example 1 (Investigation of Sodium Benzoate as Carboxylic Acid Anion Donor)

| | Formulation No. | | | | | | | | | | | |
|---|---|---|---|---|---|---|---|---|---|---|---|---|
| | 1 | | 2 | | 3 | | 4 | | 5 | | 6 | |
| | Weight | | Weight | | Weight | | Weight | | Weight | | Weight | |
| | mg | | mg | | mg | | mg | | mg | | mg | |
| Peramivir trihydrate | 69.9 | | 69.9 | | 69.9 | | 69.9 | | 69.9 | | 69.9 | |
| Sodium benzoate | 26.4 | | 52.7 | | 79.1 | | 26.4 | | 52.7 | | 79.1 | |
| Magnesium chloride | 0 | | 0 | | 0 | | 8.0 | | 8.0 | | 8.0 | |
| (hexahydrate) | | | | | | | | | | | | |
| Ultrapure water | 0.80 | ml | 0.80 | ml | 0.80 | ml | 0.80 | ml | 0.80 | ml | 0.80 | ml |
| Volume finalized by | 1.0 | ml | 1.0 | ml | 1.0 | ml | 1.0 | ml | 1.0 | ml | 1.0 | ml |

adding water

Preparation process: The formulation amount of sodium benzoate was weighed and ultrasonically vibrated in ultrapure water until dissolved, the pH of the resulting solution was regulated with a 0.1M HCl or 5M NaOH solution to 7.4±0.2, a formulation amount of peramivir trihydrate was added, followed by stirring in a water bath at 80° C. until the solution became clear, and then the solution was maintained at the temperature and vibrated for 5 minutes. The resulting solution was stirred in a water bath at room temperature to cool down, a formulation amount of magnesium chloride hexahydrate was added, stirred and dissolved, and ultrapure water was added to make up to the formulation volume. The resulting solution was filtered through a 0.22 μm filter membrane, followed by filling. The filled solution was then lyophilized in a half-capped state to prepare a lyophilized formulation, which was resolubilized with water for injection to the pre-lyophilized concentration prior to use.

Test results: the solution before lyophilization was placed at room temperature and shielded from light, a large amount of precipitate was observed in all the formulated formulations within 1 h, and the solutions became apparently cloudy.

### Example 1

Formulation by using different types of anion donors with different molar ratios:

| | Formulation No. | | | | | | | | | | | |
|---|---|---|---|---|---|---|---|---|---|---|---|---|
| | 1 | | 2 | | 3 | | 4 | | 5 | | 6 | |
| | Weight | | Weight | | Weight | | Weight | | Weight | | Weight | |
| | mg | | mg | | mg | | mg | | mg | | mg | |
| Peramivir trihydrate | 69.9 | | 69.9 | | 69.9 | | 69.9 | | 69.9 | | 69.9 | |
| Tromethamine | 38.1 | | 38.1 | | 38.1 | | 38.1 | | 38.1 | | 38.1 | |
| 2-hydroxy-3-naphthoic acid | 34.4 | | 86 | | — | | — | | — | | — | |
| 1-hydroxy-2-naphthoic acid | — | | — | | 68.8 | | — | | — | | — | |
| Pamoic acid | — | | — | | — | | 56.8 | | 71 | | 142 | |
| Magnesium sulfate | 4.8 | | 4.8 | | 4.8 | | 4.8 | | 4.8 | | 4.8 | |
| Ultrapure water | 0.80 | ml | 0.80 | ml | 0.80 | ml | 0.80 | ml | 0.80 | ml | 0.80 | ml |
| Volume finalized by adding water | 1.0 | ml | 1.0 | ml | 1.0 | ml | 1.0 | ml | 1.0 | ml | 1.0 | ml |

Preparation process: The formulation amounts of the anion donor and tromethamine were weighed and ultrasonically vibrated in ultrapure water until dissolved, the pH of the resulting solution was regulated with a 0.1M HCl or 5M NaOH solution to 7.4±0.2, a formulation amount of peramivir trihydrate was added, followed by stirring in a water bath at 75° C. until the solution became clear, and then the solution was maintained at the temperature and vibrated for 5 minutes. The resulting solution was stirred in a water bath at room temperature to cool down, a formulation amount of magnesium sulfate was added, stirred and dissolved, and ultrapure water was added to make up to the formulation volume. The resulting solution was filtered through a 0.22 μm filter membrane, followed by filling. The filled solution was then lyophilized in a half-capped state to prepare a lyophilized formulation, which was resolubilized with water for injection to the pre-lyophilized concentration prior to use.

### Example 2

Formulation by using different types of cation donors with different molar ratios:

| | Formulation No. | | | | | | | | | | | | | |
|---|---|---|---|---|---|---|---|---|---|---|---|---|---|---|
| | 1 | | 2 | | 3 | | 4 | | 5 | | 6 | | 7 | |
| | Weight | | Weight | | Weight | | Weight | | Weight | | Weight | | Weight | |
| | mg | | mg | | mg | | mg | | mg | | mg | | mg | |
| Peramivir trihydrate | 69.9 | | 69.9 | | 69.9 | | 69.9 | | 69.9 | | 69.9 | | 69.9 | |
| Pamoic acid | 63.9 | | 63.9 | | 63.9 | | 63.9 | | 63.9 | | 63.9 | | 63.9 | |
| Meglumine | 17.9 | | 17.9 | | 17.9 | | 17.9 | | 17.9 | | 17.9 | | 17.9 | |
| Magnesium sulfate | 4.8 | | 9.6 | | — | | — | | — | | — | | — | |
| Magnesium chloride | — | | — | | 5 | | 10 | | — | | — | | — | |
| Zinc chloride | — | | — | | — | | — | | 5.8 | | — | | — | |
| Anhydrous calcium | — | | — | | — | | — | | — | | 1.1 | | — | |
| chloride | | | | | | | | | | | | | | |
| Ultrapure water | 0.80 | ml | 0.80 | ml | 0.80 | ml | 0.80 | ml | 0.80 | ml | 0.80 | ml | 0.80 | ml |
| Volume finalized by | 1.0 | ml | 1.0 | ml | 1.0 | ml | 1.0 | ml | 1.0 | ml | 1.0 | ml | 1.0 | ml |

adding water

Preparation process: The formulation amounts of pamoic acid and meglumine were weighed and ultrasonically vibrated in ultrapure water until dissolved, the pH of the resulting solution was regulated with a 0.1M HCl or 5M NaOH solution to 7.4±0.2, a formulation amount of peramivir trihydrate was added, followed by stirring in a water bath at 75° C. until the solution became clear, and then the resulting solution was maintained at the temperature and vibrated for 5 minutes. The solution was stirred in a water bath at room temperature to cool down, a formulation amount of the cation donor was added, stirred and dissolved, and ultrapure water was added to make up to the formulation volume. The resulting solution was filtered through a 0.22 μm filter membrane, followed by filling. The filled solution was then lyophilized in a half-capped state to prepare a lyophilized formulation, which was resolubilized with water for injection to a pre-lyophilized concentration prior to use.

### Test Results

| | Formulation No. | | | | | | |
|---|---|---|---|---|---|---|---|
| | 1 | 2 | 3 | 4 | 5 | 6 | 7 |
| Osmotic pressure (mOSM) | 448 | 502 | 462 | 525 | 360 | 371 | 365 |
| pH of drug solution | 7.49 | 7.44 | 7.53 | 7.51 | 7.27 | 7.37 | 7.45 |
| Z-average particle size (nm) | 4.74 | 3.15 | 2.92 | 3.05 | 4.18 | 3.90 | 106.55 |

### Example 3

Formulation by using different active ingredients (in different concentrations):

| | Formulation No. | | | | | | | | | | | |
|---|---|---|---|---|---|---|---|---|---|---|---|---|
| | 1 | | 2 | | 3 | | 4 | | 5 | | 6 | |
| | Weight | | Weight | | Weight | | Weight | | Weight | | Weight | |
| | mg | | mg | | mg | | mg | | mg | | mg | |
| Peramivir trihydrate | 46.6 | | 69.9 | | 93.2 | | 116.5 | | — | | — | |
| Zanamivir | — | | — | | — | | — | | 24.3 | | — | |
| Laninamivir | — | | — | | — | | — | | — | | 25.3 | |
| Pamoic acid | 63.9 | | 63.9 | | 63.9 | | 95.9 | | 63.9 | | 63.9 | |
| Sodium hydroxide | 14.6 | | 14.6 | | 14.6 | | 14.6 | | 14.6 | | 14.6 | |
| Magnesium sulfate | 4 | | 4 | | 4 | | 4 | | 4 | | 4 | |
| Ultrapure water | 0.80 | ml | 0.80 | ml | 0.80 | ml | 0.80 | ml | 0.80 | ml | 0.80 | ml |
| Volume finalized by adding water | 1.0 | ml | 1.0 | ml | 1.0 | ml | 1.0 | ml | 1.0 | ml | 1.0 | ml |

Preparation process: The formulation amounts of pamoic acid and sodium hydroxide were weighed and ultrasonically vibrated in ultrapure water until dissolved, the pH of the resulting solution was regulated with a 0.1M HCl or 5M NaOH solution to 7.4±0.2, a formulation amount of the active ingredient was added, followed by stirring in a water bath at 90° C. until the solution became clear, and then the resulting solution was maintained at the temperature and vibrated for 5 minutes. The solution was stirred in a water bath at room temperature to cool down, a formulation amount of the cation donor was added, stirred and dissolved, and ultrapure water was added to make up to the formulated volume. The resulting solution was filtered through a 0.22 μm filter membrane, followed by filling. The filled solution was then lyophilized in a half-capped state to prepare a lyophilized formulation, which was resolubilized with water for injection to the pre-lyophilized concentration prior to use.

### Example 4

### Investigation on Stability at Different pH Values

| | Formulation No. | | | | |
|---|---|---|---|---|---|
| | 1 | 2 | 3 | 4 | 5 |
| | Weight | Weight | Weight | Weight | Weight |
| | mg | mg | mg | mg | mg |
| Peramivir trihydrate | 58.2 | 58.2 | 58.2 | 58.2 | 58.2 |
| Pamoic acid | 29.5 | 29.5 | 29.5 | 29.5 | 29.5 |
| Sodium hydroxide | 14.6 | 14.6 | 14.6 | 14.6 | 14.6 |
| Magnesium chloride | 5 | 5 | 5 | 5 | 5 |
| Ultrapure water | 0.80 ml | 0.80 ml | 0.80 ml | 0.80 ml | 0.80 ml |
| Regulated pH | 6.0 | 7.8 | 8.3 | 8.8 | 9.6 |
| Volume finalized | 1.0 ml | 1.0 ml | 1.0 ml | 1.0 ml | 1.0 ml |
| by adding water | | | | | |

Preparation process: The formulation amounts of pamoic acid and sodium hydroxide were weighed and ultrasonically vibrated in ultrapure water until dissolved, the formulation amount of the active ingredient was added, the mixture was stirred in a water bath at 75° C. until the solution became clear, and then the resulting solution was maintained at the temperature and vibrated for 5 minutes. The solution was stirred in a water bath at room temperature to cool down, the formulation amount of the cation donor was added, stirred and dissolved, the pH of the solution was regulated with a 0.1M HCl or 5M NaOH solution to 6.0, 7.8, 8.3, 8.8, and 9.6, respectively, and ultrapure water was added to make up to the formulated volume. The resulting solution was filtered through a 0.22 μm filter membrane, followed by filling. The filled solution was then lyophilized in a half-capped state to prepare a lyophilized formulation, which was resolubilized with water for injection to the pre-lyophilized concentration prior to use.

Test results: After resolubilization with water for injection, Formulation 1, Formulation 2, and Formulation 3 were clear and transparent, Formulation 4 was slightly cloudy, and Formulation 5 was apparently cloudy. After standing for 6 h, Formulation 1, Formulation 2, and Formulation 3 were clear and transparent. After standing for 20 h, Formulation 2 and Formulation 3 were clear and transparent, and Formulation 1 was slightly cloudy.

### Performance Test

Unless otherwise specified, the following high performance liquid chromatography conditions were used to detect the amount of peramivir:

### Analysis Conditions for Test Samples

Detection system: HPLC-UV (Shimadzu SPD-10AVP, LC-20AD)
Liquid chromatography column: Ultimate XB-C18 4.6×150 mm 5 μm
Column temperature: 35° C.
Mobile phase A: 10 mM potassium dihydrogen phosphate (pH=3.0)
Mobile phase B: Acetonitrile
Initial phase: 30% phase B
Needle wash solution: 50% acetonitrile aqueous solution
Flow rate: 1.000 mL/min
Detection wavelength: 280 nm
Liquid phase ratio:

| Time | Module | Event | Parameter |
|---|---|---|---|
| 7.00 | Controller | Stop | — |

### Analysis and Detection Conditions for Biological Samples

Detection system: LCMS-8045
Liquid chromatography column: Luna Omega C18 50×2.1 mm, 1.6 μm (SN: H20-064432)
Mobile phase A: 0.1% formic acid aqueous solution (pH=5.3)
Mobile phase B: Methanol
Needle wash solution:Methanol:Isopropanol:Water (1:1:1)
Flow rate: 0.5 mL/min
Column temperature: 40° C.
Autosampler temperature: 4° C.
Liquid phase ratio:

| Time | Unit | Command | Value |
|---|---|---|---|
| 0.50 | Column oven | CTO.BVL | 1 |
| 1.50 | Pump | Pump B Conc. | 95 |
| 1.80 | Column oven | CTO.BVL | 0 |
| 2.00 | Pump | Pump B Conc. | 95 |
| 2.01 | Pump | Pump B Conc. | 30 |
| 3.00 | Controller | Stop | — |

Mass spectrometry system: YQ-09-067
Ionization mode: ESI+
Scan mode: MRM

### Test of Formulation 5 of Example 1: Pharmacokinetic Study of Plasma and lung Tissue in SD Rats After Aerosol Inhalation

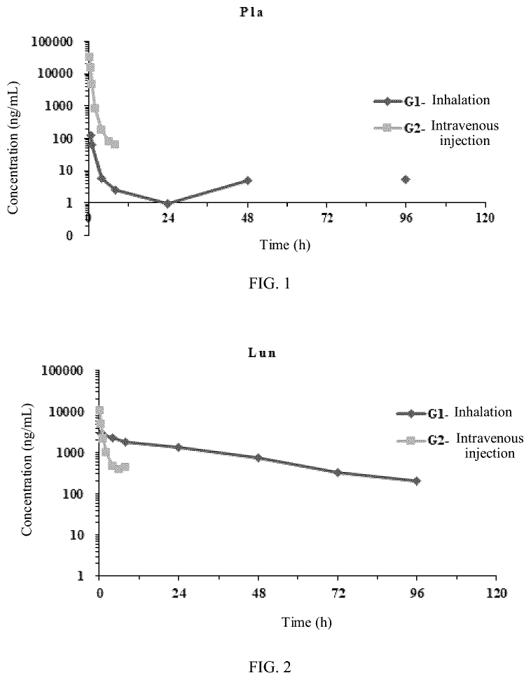

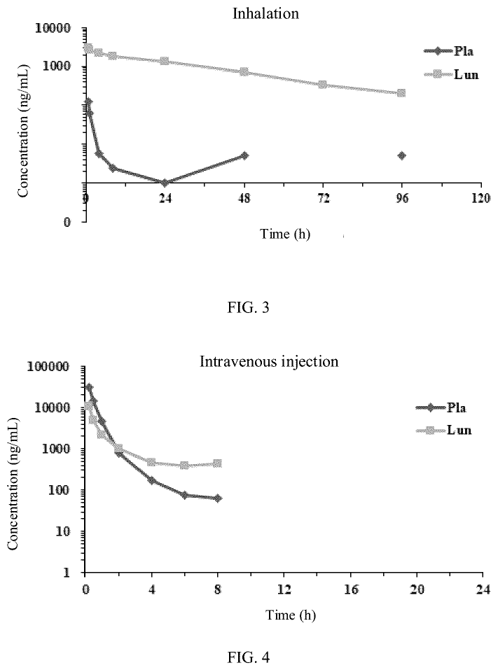

Test method: 45 male rats (SPF-grade SD rats, the range of body weights: 180 g to 220 g at the time of purchase, purchased from Zhejiang Vital River Laboratory Animal Technology Co., Ltd.) were randomly divided into two groups. The two groups were administrated with a 4.50 mg/kg peramivir inhalation solution (the solution was inhaled, 24 rats in Group 1) and a 25 mg/kg peramivir injection solution (the solution was intravenously injected, 21 rats in Group 2), respectively, and blood samples and lung tissues were collected at 0.5, 1, 4, 8, 24, 48, 72 and 96 h after administration and at 0.25, 0.5, 1, 2, 4, 6 and 8 h after administration, respectively. The concentrations of peramivir in the plasma and lung tissues were determined by LC-MS/MS and the pharmacokinetic parameters were calculated by using WinNonlin 8.2. The drug concentration-time profiles of the two experimental groups are shown in FIGS. 1 to 4.

Test results: the drug exposure in plasma in Group 1 (inhalation group) was lower than that in Group 2 (intravenous injection group), but the drug exposure in lung tissues in Group 1 was higher than that in Group 2, and the peramivir inhalation solution via aerosol inhalation exhibited the advantages of high local exposure, low systemic circulation exposure and longer half-life. The main pharmacokinetic parameters are shown in the following table:

| | | Group 1 | Group 2 |
|---|---|---|---|
| | | (peramivir | (peramivir |
| | | inhalation | injection |
| | | solution) | solution) |
| Plasma/Tissue | Parameters | 4.50 mg/kg | 25 mg/kg |
| Plasma (Pla) | AUC_{(0-24 h)}(ng/mL*h) | 418 | 27100 |
| | t_{1/2z}(h) | 97.6 | 1.62 |
| | Tₘₐₓ(h) | 0.500 | 0.250 |
| | Cₘₐₓ(ng/mL) | 122 | 31600 |
| Lung (Lun) | AUC_{(0-24 h)}(ng/g*h) | 86300 | 12600 |
| | t_{1/2z}(h) | 26.7 | 2.31 |
| | Tₘₐₓ(h) | 0.500 | 0.250 |
| | Cₘₐₓ(ng/g) | 2970 | 10600 |

### Test of Formulation 5 of Example 1: Histopathological and Toxicokinetic Studies in SD Rats After Aerosol Inhalation

Test method: A small-animal single concentration oral and nasal exposure system and an impact type liquid aerosol generator were used. The parameters for a low dose group were set with an aerosol flow rate of 10 L/min, a dilution flow rate of 0 L/min, and a pumping flow rate of 6 L/min, and the parameters for a high dose group were set with an aerosol flow rate of 16 L/min, a dilution flow rate of 0 L/min, and a pumping flow rate of 12 L/min. Sampling was performed after the aerosol concentration was stabilized, and after the sampling, the animals were exposed to virus for 4 h, and the concentration was detected every 1 h at the sampling port. The animals were sequentially removed after the virus exposure was completed, and the particle size distribution was detected by using an aerodynamic particle size analyzer.

40 healthy animals (SPF-grade SD rats, the range of body weights: Female 186.4 g to 229.1 g, Male 217.2 g to 248.9 g at the time of grouping, purchased from Zhejiang Vital River Laboratory Animal Technology Co., Ltd.) were selected and randomly divided into two groups according to sex and body weight, 20 animals in each group, including 6 animals for the main experimental group and 14 animals for the toxicokinetic (TK) satellite group, half male and half female in each group. The animals were administrated once by oral and nasal exposure and inhalation for 4 h and were observed for 7 days. The animals of the main experimental group were observed for clinical symptoms every day during the observation period, weighed on Day 1, Day 2, Day 4 and Day 7, and dissected, sampled and fixed for histopathological examination on Day 8. For the animals of the TK satellite group, blood samples were collected at 0.5, 1, 2, 4, 6, 8, 24, 36, 48, and 72 h after administration, and the lung tissues and the lavage fluids were collected at 0.5, 4, 8, 24, 36, 48, and 72 h after administration for detection and analysis.

Test results: The actual administered dose was 67.30 mg/kg in the low dose group and 125.16 mg/kg in the high dose group. During the observation period, no abnormal clinical symptoms were observed in the animals of the main experimental group, and the body weight of the animals in the high dose group had a transient decrease on the day of administration, and then recovered on the next day and increased steadily. Histopathological examination results showed that, for both the low dose group and the high dose group, very slight to mild neutrophil infiltration and focality occurred in the lungs of the animals, and very slight inflammatory cell infiltration and focality of the epiglottic lamina propria and very slight edema occurred in the throats of the animals. The number and frequency of lesions in the low dose group were comparable with those in the high dose group, and there was no dose-response relationship.

TABLE 1
Summary of histopathological examination
results - abnormal lesions

| | | | Low dose | | High dose | |
|---|---|---|---|---|---|---|
| Name | | | group | | group | |
| of | Microscopic examination | | Female | Male | Female | Male |
| organs | of lesions | | n = 3 | n = 3 | n = 3 | n = 3 |
| Lung | Neutrophil infiltration | + | 2 | 0 | 1 | 0 |
| | and focality | 2+ | 0 | 0 | 1 | 0 |
| Throat | Inflammatory cell | + | 0 | 2 | 1 | 1 |
| | infiltration and | | | | | |
| | focality of epiglottic | | | | | |
| | lamina propria Edema | + | 0 | 2 | 1 | 1 |

Note:
Lesion severity grading, “+” for very slight, “2+” for mild, “3+” for moderate, and “4+” for severe.

After the administration of 67.30 mg/kg and 125.16 mg/kg peramivir inhalation solutions, respectively, the plasma of the female rats showed AUC_{0-72 h} of 6,040 ng/ml*h and 41,000 ng/ml*h, respectively; Cₘₐₓ of 1,060 ng/ml and 5,100 ng/mL, respectively; Tₘₐₓ of 0.500 h and 2.00 h, respectively; and T_{1/2} of 7.77 h and 12.3 h, respectively. The plasma of the male rats showed AUC0-72 h of 4,360 ng/mL*h and 25,100 ng/ml*h, respectively; Cₘₐₓ of 715 ng/ml and 1,520 ng/mL, respectively; Tₘₐₓ of 0.500 h and 8.00 h, respectively; and T_{1/2} of 11.0 h and 8.84 h, respectively.

The lung tissues of the female rats showed AUC_{0-72 h} of 202,000 ng/ml*h and 222,000 ng/ml*h, respectively; Cₘₐₓ of 6,290 ng/ml and 8,330 ng/mL, respectively; Tₘₐₓ of 0.500 h and 4.00 h, respectively; and T_{1/2} of 29.7 h and 21.0 h, respectively. The lung tissues of the male rats showed AUC_{0-72 h} of 210,000 ng/ml*h and 271,000 ng/ml*h, respectively; Cₘₐₓ of 7,879 ng/ml and 10,400 ng/mL, respectively; Tmax of 0.500 h and 4.00 h, respectively; and T_{1/2} of 24.4 h and 28.0 h, respectively.

The lavage fluid of the female rats showed AUC_{0-72 h} of 4,780 ng/ml*h and 7,940 ng/ml*h, respectively; Cₘₐₓ of 598 ng/mL and 423 ng/ml, respectively; Tₘₐₓ of 0.500 h for both; and T_{1/2} of 38.3 h and 22.7 h, respectively. The lavage fluid of the male rats showed AUC_{0-72 h} of 4,350 ng/ml*h and 7,310 ng/ml*h, respectively; Cₘₐₓ of 574 ng/ml and 393 ng/mL, respectively; Tₘₐₓ of 0.500 h for both; and T_{1/2} of 27.3 h and 25.9 h, respectively.

The toxicokinetic results showed that, for both the low dose group and the high dose group, the exposure in plasma was much lower than the exposure in the lung tissue, and the exposure in plasma was slightly higher in the females than in the males. The plasma exposure ratio between the low dose group and the high dose group were 6.79 for the females and 5.76 for the males, both higher than the dose ratio 1.86; there was no significant dose relationship between the lung tissue drug exposures in the low dose group and the high dose group, and there was no gender difference.

While the embodiments described herein have been described with reference to specific examples, it should be understood that various modifications and changes may be made thereto by those skilled in the art without departing from the scope and ideas of the present disclosure.

1. A pharmaceutical formulation, wherein raw materials for formulating the pharmaceutical formulation comprise:
an active ingredient which is one selected from the group consisting of peramivir, zanamivir, laninamivir, and hydrates or solvates thereof;
a carboxylic acid anion donor which is at least one selected from the group consisting of pamoic acid and hydroxynaphthoic acid; and
a first pH regulator which is an alkaline pH regulator.

2. The pharmaceutical formulation according to claim 1, wherein the hydroxynaphthoic acid is one or more selected from the group consisting of 2-hydroxy-1-naphthoic acid, 3-hydroxy-1-naphthoic acid, 4-hydroxy-1-naphthoic acid, 5-hydroxy-1-naphthoic acid, 6-hydroxy-1-naphthoic acid, 1-hydroxy-2-naphthoic acid, 2-hydroxy-3-naphthoic acid, and 8-hydroxy-2-naphthoic acid; and optionally, the hydroxynaphthoic acid is 2-hydroxy-3-naphthoic acid.

3. The pharmaceutical formulation according to claim 1, wherein the first pH regulator is one or more selected from the group consisting of sodium hydroxide, potassium hydroxide, diethylamine, triethylamine, ethylenediamine, sodium bicarbonate, sodium carbonate, trometamol, and meglumine.

4. The pharmaceutical formulation according to claim 1, wherein a molar ratio of the active ingredient to a carboxyl group in the carboxylic acid anion donor is 1:(0.5 to 3), or 1:(0.7 to 2).

5. The pharmaceutical formulation according to claim 1, wherein the raw materials further comprise a cation donor;
optionally, the cation donor is one or more selected from the group consisting of inorganic salts of magnesium ions, calcium ions, zinc ions, potassium ions, and sodium ions, trometamol, meglumine, and ethylenediamine; or the cation donor is one or more selected from the group consisting of magnesium chloride, magnesium sulfate, calcium chloride, and zinc chloride, and the cation donor is the same as or different from the first pH regulator.

6. The pharmaceutical formulation according to claim 5, wherein a molar ratio of the active ingredient:a carboxyl group in the carboxylic acid anion donor:a cation in the cation donor is 1:(0.5 to 3):(0 to 5), or 1:(0.7 to 2):(0.2 to 2).

7. The pharmaceutical formulation according to claim 6, wherein the raw materials comprise:
the active ingredient which is one selected from the group consisting of peramivir, zanamivir, laninamivir and hydrates or solvates thereof;
the carboxylic acid anion donor which is one or more selected from the group consisting of 2-hydroxy-3-naphthoic acid, 1-hydroxy-2-naphthoic acid, and pamoic acid;
the first pH regulator which is one or more selected from the group consisting of sodium hydroxide, potassium hydroxide, diethylamine, etbylenediamine, triethylamine, sodium bicarbonate, sodium carbonate, trometamol, and meglumine; and
optionally, a cation donor which is one or more selected from the group consisting of magnesium chloride, magnesium sulfate, calcium chloride, and zinc chloride.

8. The pharmaceutical formulation according to claim 1, wherein the raw materials further comprise a second pH regulator which is one or more selected from the group consisting of sodium hydroxide, potassium hydroxide, diethylamine, triethylamine, ethylenediamine, sodium bicarbonate, sodium carbonate, trometamol, meglumine, hydrochloric acid, sodium citrate, citric acid, sodium dihydrogen phosphate, disodium hydrogen phosphate, and phosphoric acid.

9. The pharmaceutical formulation according to claim 1, wherein the pharmaceutical formulation is an aqueous pharmaceutical formulation or a lyophilized pharmaceutical formulation, optionally, the aqueous pharmaceutical formulation is a liquid inhalation formulation, an aqueous injection solution, a spray, or an aerosol; optionally, the aqueous pharmaceutical formulation is an oral aerosol inhalation formulation.

10. The pharmaceutical formulation according to claim 9, wherein the pharmaceutical formulation is an aqueous pharmaceutical formulation, wherein a concentration of the active ingredient is up to 100 mg/ml, and optionally the concentration of the active ingredient ranges from 20 mg/ml to 100 mg/ml; or the pharmaceutical formulation is a lyophilized pharmaceutical formulation, and in an aqueous soultion reformulated from the lyophilized pharmaceutical formulation with water, a concentration of the active ingredient is up to 100 mg/ml, and optionally the concentration of the active ingredient ranges from 20 mg/ml to 100 mg/ml.

11. The pharmaceutical formulation according to claim 9, wherein the aqueous pharmaceutical formulation or the aqueous soultion reformulated from the lyophilized pharmaceutical formulation with water has a pH value of 5.5 to 8.5 or 6.5 to 8.0.

12. The pharmaceutical formulation according to claim 9, wherein the pharmaceutical formulation is a lyophilized pharmaceutical formulation containing a lyophilization excipient; optionally, the lyophilization excipient is one or more selected from the group consisting of sodium chloride, glucose, glycine, cysteine, and lysine; or the lyophilization excipient is one selected from the group consisting of sodium chloride, glucose, and glycine; optionally, the lyophilized pharmaceutical formulation contains 0 wt % to 5 wt % of the lyophilization excipient.

13. The pharmaceutical formulation according to claim 1, wherein a route of administration of the pharmaceutical formulation is selected from the group consisting of intravenous administration, oral aerosol inhalation, nasal administration, subcutaneous administration, intradermal administration, and intramuscular administration, or is oral aerosol inhalation.

14. A preparation method of the pharmaceutical formulation according to claim 1, comprising the following steps:
(1) dissolving the carboxylic acid anion donor and the alkaline pH regulator in water; and
(2) adding the active ingredient into the aqueous solution obtained in step (1).

15. The preparation method according to claim 14, further comprising:
(3) adding the cation donor after the active ingredient is added.

16. The preparation method according to claim 14, further comprising, by using a second pH regulator, adjusting a pH value of the aqueous solution obtained in step (1) to 7 or higher or to 7 to 9, and/or adjusting a pH value of the aqueous solution obtained in step (2) to 5.5 to 8.5 or 6.5 to 8.0,
wherein the second pH regulator is one or more selected from the group consisting of sodium hydroxide, potassium hydroxide, diethylamine, triethylamine, ethylenediamine, sodium bicarbonate, sodium carbonate, trometamol, meglumine, hydrochloric acid, sodium citrate, citric acid, sodium dihydrogen phosphate, disodium hydrogen phosphate, and phosphoric acid.

17. The preparation method according to claim 14, further comprising a step of aseptically filling and/or lyophilizing the aqueous solution obtained in step (2) to obtain a lyophilized pharmaceutical formulation.

18. A method for preventing or treating a disease caused by an influenza virus, e.g., an acute respiratory infectious disease caused by influenza A virus or influenza B virus, comprising administrating the pharmaceutical formulation according to claim 1 to a subject in need thereof.
